(19) United States
(12) Patent Application Publication
(10) Pub. No.: US 2015/0093765 A1
(43) Pub. Date: Apr. 2, 2015

(54) METHOD OF PROGNOSIS

(71) Applicants: Region Midtjylland, Viborg (DK); Aarhus Universitet, Arhus C (DK)

(72) Inventors: Søren Moestrup, Arhus N (DK); Ruth Frikke-Schmidt, Alsgarde (DK); Anne Tybjaerg-Hansen, Gentofte (DK); Holger J. Moller, Risskov (DK)

(21) Appl. No.: 14/570,194

(22) Filed: Dec. 15, 2014

Related U.S. Application Data
(63) Continuation of application No. 13/501,157, filed on Jun. 25, 2012, now abandoned.

(30) Foreign Application Priority Data
Oct. 12, 2009 (DK) PA 2009 70156

(51) Int. Cl. G01N 33/68

(52) U.S. Cl. CPC G01N 33/6872; G01N 2800/042; G01N 2800/085; G01N 2800/52; G01N 2333/70596; G01N 2800/50
USPC 435/7.21; 436/501; 436/86; 435/29; 204/450

(57) ABSTRACT

The present invention relates to the use of soluble CD163 as a prognostic marker for the assessment of the risk for contracting a disorder, in particular for contracting diabetes and/or a liver disorder. The invention also relates to the use of CD163 as a prognostic marker for assessing lifetime expectancy.

All patent and non-patent references cited in the application are hereby incorporated by reference in their entirety.

### FIELD OF INVENTION

The present invention relates to the use of soluble CD163 as a prognostic marker for the assessment of the risk for contracting a disorder, in particular for contracting diabetes and/or a liver disorder. The invention also relates to the use of CD163 as a prognostic marker for assessing lifetime expectancy.

### BACKGROUND OF INVENTION

The frequency of diabetes and other life-style related diseases is increasing dramatically due to the global obesity epidemic. As many life-style related diseases can be combated by change in life-style or by early drug treatment, there is a need for identifying subjects that are at high risk of contracting e.g. diabetes, non-alcoholic fatty liver, and low-grade systemic inflammation. Today, the likelihood of contracting such life-style related disorders is predicted by measuring BMI, blood-pressure, fasting blood glucose and other traditional clinical investigations. Recently the use of serum-markers has attracted interest. One such serum-marker is C-reactive protein (CRP), an acute-phase protein and a sensitive marker for systemic inflammation. Recent research suggests that patients with elevated basal levels of CRP are at an increased risk of contracting diabetes, hypertension and cardiovascular disease. The studies that have led to the use of CRP as a predictive marker have shown an association between elevated CRP and an existing clinical condition such as overweight. See e.g. Visser et al [Visser M, et al: Low-grade systemic inflammation in overweight children; Pediatrics 2001 107 e13].

Thus, a need for identifying predictive markers that can be used to identify subjects at increased risk of contracting a disease or disorder, before there are any other signs of the disorder, is clearly present. The haptoglobin-hemoglobin receptor CD163 [Kristiansen M et al: Identification of the haemoglobin scavenger receptor; Nature 2001 409 198-201] is closely related to macrophage activation, since substantial amounts of the extracellular part of the molecule, soluble CD163 (sCD163), are shed to blood upon inflammatory activation of macrophages [Moller H J, et al: Identification of the hemoglobin scavenger receptor/CD163 as a natural soluble protein in plasma; Blood 2002 99 378-80; Weaver L K, et al: Pivotal Advance: Activation of cell surface Toll-like receptors causes shedding of the hemoglobin scavenger receptor CD163. J Leukoc Biol 2006 80 26-35]. CD163 is exclusively expressed on macrophages and monocytes and highly expressed in human adipose tissue-macrophages and on Kupffer cells [Zeyda M, et al: Human adipose tissue macrophages are of an anti-inflammatory phenotype but capable of excessive pro-inflammatory mediator production; Int J Obes 2007 31 1420-1428]. It is shed to the blood in response to macrophage Toll-like receptor activation [Weaver L K, et al: Pivotal Advance: Activation of cell surface Toll-like receptors causes shedding of the hemoglobin scavenger receptor CD163; J Leukoc Biol 2006 80 26-35].

Soluble and membrane-bound CD163 have been shown to be elevated in a number of clinical conditions [Moestrup et al: CD163: a regulated hemoglobin scavenger receptor with a role in the anti-inflammatory response; Ann Med 2004 36 347-354]. U.S. Pat. No. 7,144,710 (Trustees of Dartmouth College) describes a general correlation between sCD163 and inflammation and documents that sCD163 may be used as an acute-phase marker of an inflammatory response.

WO 02/32941 (Proteopharma) discloses that sCD163 may be 5-10 times elevated in patients in a hematologic hospital unit compared to the level in healthy blood-donors. The publication also describes the use of sCD163 as a diagnostic marker for hemolytic patients and patients with haematological diseases and as an acute marker for inflammation and immunodeficiency. The publication further describes methods for detection of sCD163, including Elisa, RIA, chromatography, electrophoresis, and mass-spectrometry.

Bleesing et al [Bleesing et al: The diagnostic significance of soluble CD163 and soluble interleukin-2 receptor alpha-chain in macrophage activation syndrome and untreated new-onset systemic juvenile idiopathic arthritis; Arthritis & Rheumatism 2007 56 965-971], discloses that sCD163 may be used as a diagnostic maker for macrophage activation syndrome in patients with juvenile idiopathic arthritis. The reference speculates whether sCD163 may be used to identify subclinical macrophage-activation-syndrome in patients with juvenile idiopathic arthritis. The reference concerns prognosis in individuals that have already been diagnosed with juvenile idiopathic arthritis.

Møller et al [Møller H J et al: Biological variation of soluble CD163; Scand J Clin Lab Invest 2003 63 15-21], concerns measurement of sCD163 in individuals at different times of the day. It is concluded that CD163 levels are very individual and that the variation between individuals is much larger than within individuals. The reference does not associate CD163 levels to any clinical conditions.

### SUMMARY OF INVENTION

The present invention relates to the novel use of the CD163 protein as a prognostic marker for predicting the risk of contracting a disorder. By obtaining a biological sample from an individual, the likelihood of contracting said disorder may be predicted according to the level of CD163 in said sample. More specifically, the invention relates to the finding that an increase in blood levels of sCD163 correlates with an increased risk of contracting a disorder.

In a first aspect, the present invention relates to a method for assessing the likelihood of contracting a disorder, said method comprising determining the amount of CD163 in a biological sample from an individual wherein a high level of CD163 is indicative of an increased likelihood.

Ability to predict the likelihood of contracting any disease before overt signs of said disease are present is of utmost importance as prophylactic treatment may be easier, gentler to the individual, and in many cases also less expensive to both the individual and society. The present invention relates to prevalent disorders that may be prevented by increased physical exercise and by altered diet, for example reduced intake of fat, sugar and alcohol, if discovered early. The present invention may therefore contribute to improved life quality for large groups of the population in the developed countries as well as severely reduce society expenses in the health care system.

In a preferred embodiment, the present invention relates to the use of CD163 to predict the risk of contracting diabetes, more specifically type 2 diabetes.

In another preferred embodiment, the present invention relates to the use of CD163 to predict the risk of contracting liver diseases, more specifically fatty liver disease, most specifically hepatic steatosis.

In yet another preferred embodiment, the present invention relates to the use of CD163 to predict an individual's lifetime expectancy.

In another aspect, the present invention relates to a kit comprising at least one binding protein, said binding protein being linked to a solid support. In a preferred embodiment, said kit comprising another binding protein, wherein said binding protein being covalently linked to a detection moiety. In another preferred embodiment, said solid support is a microparticle. In another preferred embodiment, said microparticle is detectable in a sample upon CD163 binding. In yet another preferred embodiment, said binding protein is an antibody directed against CD163.

In another aspect, the invention relates to metformin for use in a method of prophylactic treatment of type 2 diabetes, in a subject having a high CD163 level.

In another aspect, the invention relates to a compound selected from the group consisting of glucose-dependent insulinotropic polypeptide (GIP), nicotinic acid, pioglitazone, ramipril, curcumin, fructanes, acarbose, vitamin D, butyrate, thiazolidinediones, mesalazine, salsalate, advair, flovent, atenolol, resveratrol and statins, for use in a method of prophylactic treatment of low grade inflammation in adipose tissue and liver, in a subject having a high sCD163 level.

The level of CD163 is particularly applicable for identifying the subgroups of overweight individuals with the highest risk of developing type 2 diabetes. Therefore, in yet another aspect, the invention relates to gastric bypass procedure for use in a method of intensive treatment of type 2 diabetes in overweight individuals having an increased CD163 level as defined herein. In a time of limited health resources, identification of high risk groups that will benefit most from expensive procedures, such as for example gastric bypass procedure, is of utmost importance.

### FIGURE LEGENDS

FIG. 1

Amino acid sequence of CD163 (Swissprot-Uniprot accession number Q86VB7). The full amino acid sequence of CD163. Residues 1-41 represent the signal peptide, residues 42-1050 represent the extracellular part, 1051-1071 the transmembrane domain and 1052-1156 the cytoplasmic domain. Residues 42-1050 are highlighted as this sequence represent the extracellular, and therefore potentially, soluble part of the molecule.

FIG. 2

Cumulative incidence of type 2 diabetes events and fatty liver disease as a function of age by plasma sCD163 levels in the general population.

During 16 years of follow-up, 511 of 8,694 event-free participants developed diabetes. The cumulative incidence of diabetes as a function of age was increased with increasing plasma sCD163 percentile categories (log-rank P for trend, <0.0001). At the age of 80 years, 10%, 20%, 34%, and 43%, respectively, of individuals with sCD163 in 34-66%, 67%-90%, 91%-95%, and 96%-100% categories had type 2 diabetes compared with 7% for the 0%-30% category.

During 16 years of follow-up, 136 participants developed fatty liver disease. The cumulative incidence of fatty liver disease as a function of age was increased with increasing plasma sCD163 percentile categories (log-rank P for trend, <0.0001). At the age of 80 years, 3%, 5%, 15%, and 35%, respectively, of individuals with sCD163 in 34-66%, 67%-90%, 91%-95%, and 96%-100% categories had fatty liver disease compared with 2% for the 0%-30% category.

FIG. 3

Absolute 10-year risk of type 2 diabetes according to plasma sCD163 percentile category, body mass index, sex, and age.

10 year absolute risk was highest among women and men above 50 years and with body mass index (BMI) above 25, and sCD163 in the highest percentile category (96-100%). Among overweight (BMI>25) individuals, high sCD163 (96-100% percentile group) was particularly good in detecting the group with highest risk for diabetes (4-fold absolute 10 year risk compared to the 0-33% percentile group). In other words, levels of sCD163 are very good at discriminating between the groups of individuals where overweight in particular predispose to diabetes and where overweight does not.

In respect of each of the groups, the five columns from left towards right corresponds to 0-33 percentiles, 34-66 percentiles, 67-90 percentiles, 91-95 percentiles and 96-100 percentiles, respectively.

FIG. 4

Proportion surviving as a function of age by plasma sCD163 levels in the general population.

The proportion surviving decreased with increased sCD163 percentile categories (log-rank P for trend test, <0.0001). The median survival was decreased with 13.5 years in the 96-100% percentile category compared with the 0-33% percentile category. This decrease in lifespan is greater that the lifespan loss observed in smokers (approx. 9 years).

The five graphs in the figure and in the region where the graphs are separate and when considered from above and downward correspond to 0-33%, 34-66%, 67-90%, 91-95% and 96-100%, respectively.

FIG. 5

Plasma concentrations of sCD163 in the general population according to age and sex.

### DEFINITIONS

The term CD163 used herein refers to both soluble and membrane-bound forms. CD163 is also known as CD163=Hemoglobin receptor=Haptoglobin-Hemoglobin receptor=Hemoglobin scavenger receptor=HbSR=M130=RM3/1 epitopeDiabetes. The term “sCD163”=soluble CD163=shed CD163=plasma CD163=serum CD163=circulating CD163

Liver diseases include the following ICD10 classified liver diseases:

K70 Alcoholic liver disease
Alcoholic fatty liver
Alcoholic hepatitis
Alcoholic fibrosis and sclerosis of liver
Alcoholic cirrhosis of liver
Alcoholic cirrhosis not otherwise specified (NOS)
   Alcoholic hepatic failure
NOS
acute
chronic
subacute
with or without hepatic coma
   Alcoholic liver disease, unspecified
   K71 Toxic liver disease
   Toxic liver disease with cholestasis
Cholestasis with hepatocyte injury
“Pure” cholestasis
   Toxic liver disease with hepatic necrosis
Hepatic failure (acute), (chronic) due to drugs
   Toxic liver disease with acute hepatitis
   Toxic liver disease with chronic persistent hepatitis
   Toxic liver disease with chronic lobular hepatitis
   Toxic liver disease with chronic active hepatitis
Toxic liver disease with lupoid hepatitis
   Toxic liver disease with hepatitis, not elsewhere classified
   Toxic liver disease with fibrosis and cirrhosis of liver
   Toxic liver disease with other disorders of liver
Toxic liver disease with:
focal nodular hyperplasia
hepatic granulomas
peliosis hepatis
veno-occlusive disease of liver
   Toxic liver disease, unspecified
   K72 Hepatic failure, not elsewhere classified
   hepatic:
coma NOS
encephalopathy NOS
   hepatitis:
acute
fulminant
malignant
   liver (cell) necrosis with hepatic failure
   yellow liver atrophy or dystrophy
   Acute and subacute hepatic failure
   Chronic hepatic failure
   Hepatic failure, unspecified
   K73 Chronic hepatitis, not elsewhere classified
   Chronic persistent hepatitis, not elsewhere classified
   Chronic lobular hepatitis, not elsewhere classified
   Chronic active hepatitis, not elsewhere classified
Lupoid hepatitis NEC
   Other chronic hepatitis, not elsewhere classified
   Chronic hepatitis, unspecified
   K74 Fibrosis and cirrhosis of liver
   Hepatic fibrosis
   Hepatic sclerosis
   Hepatic fibrosis with hepatic sclerosis
   Primary biliary cirrhosis
Chronic nonsuppurative destructive cholangitis
   Secondary biliary cirrhosis
   Biliary cirrhosis, unspecified
   Other and unspecified cirrhosis of liver
Cirrhosis (of liver):
NOS
cryptogenic
macronodular
micronodular
mixed type
portal
postnecrotic
   K76 Other diseases of liver
   Fatty (change of) liver, not elsewhere classified
   Chronic passive congestion of liver
Cardiac:
cirrhosis (so-called) of liver
sclerosis of liver
   Central haemorrhagic necrosis of liver
   Infarction of liver
   Peliosis hepatis
Hepatic angiomatosis
   Hepatic veno-occlusive disease
   Portal hypertension
   Hepatorenal syndrome
   Other specified diseases of liver
Focal nodular hyperplasia of liver
Hepatoptosis
   Liver disease, unspecified

The term ‘soluble’ used herein refers to the property of a solid, liquid, or gaseous chemical substance to dissolve in a liquid solvent to form a homogeneous solution. Further it refers to a compound, such as a protein, being in liquid solution as not being attached to a membrane or other anchoring or attaching moeities.

The term ‘prognostic marker’ used herein refers to the characteristic of a compound, such as a protein, that can be used to estimate the chance of contracting a disease over a period of time in the absence of therapy.

The term ‘disorder’ used herein refers to a disease or medical problem, and is an abnormal condition of an organism that impairs bodily functions, associated with specific symptoms and signs. It may be caused by external factors, such as invading organisms, or it may be caused by internal dysfunctions.

The term ‘diabetes’ used herein refers to a condition in which the body does not produce enough, or properly respond to, insulin, causing glucose to accumulate in the blood. This leads to complications such as hypoglycemia, diabetic ketoacidosis, nonketotic hyperosmolar coma, cardiovascular disease, chronic renal failure, retinal damage, which can lead to blindness, nerve damage, microvascular damage, erectile dysfunction, poor wound healing, gangrene, and possibly amputation.

The term ‘protein’ used herein refers to an organic compound, also known as a polypeptide, which is a peptide having at least, and preferably more than two amino acids. The generic term amino acid comprises both natural and non-natural amino acids any of which may be in the ‘D’ or ‘L’ isomeric form.

The term ‘biological sample’ used herein refers to any sample selected from the group, but not limited to, serum, plasma, whole blood, saliva, urine, lymph, a biopsy, semen, faeces, tears, sweat, milk, cerebrospinal fluid, ascites fluid, synovial fluid.

The term ‘binding assay’ used herein refers to any biological or chemical assay in which any two or more molecules bind, covalently or noncovalently, to each other thereby enabling measuring the concentration of one of the molecules.

The term ‘chromatographic method’ used herein refers to a collective term for the process of separating mixtures. It involves passing a mixture dissolved in a “mobile phase” through a stationary phase, which separates the analyte to be measured from other molecules in the mixture and allows it to be isolated.

The term ‘risk factor’ used herein refers to a variable associated with an increased risk of disease or infection. Risk factors are correlational and not necessarily causal, because correlation does not imply causation.

The term ‘detection moiety’ used herein refers to a specific part of a molecule, preferably but not limited to be a protein, able to bind and detect another molecule.

### DETAILED DESCRIPTION OF THE INVENTION

### CD163

The incidence of obesity and associated diseases such as diabetes 2 and fatty liver disease has increased dramatically during recent decades. As the development of said diseases may be halted if diagnosed before overt diseases have developed, a need for prognostic biomarkers is evident.

The present invention relates to the use of CD163 as a sensitive, prognostic biomarker for low grade inflammation, diabetes, liver disease and reduced life expectancy. In a preferred embodiment, the invention may enable physicians to discriminate between high and low risk diabetes groups throughout the entire age and body mass index (BMI) spectrum of a population by obtaining a biological sample from an individual, although particularly for overweight individuals over the age of 50. In another preferred embodiment, the invention relates to the finding that sCD163 plasma levels can predict the incident of type 2 diabetes and fatty liver disease before overt disease develops. In yet another preferred embodiment, the invention relates to the finding that the levels of sCD163 can predict reduced life expectancy.

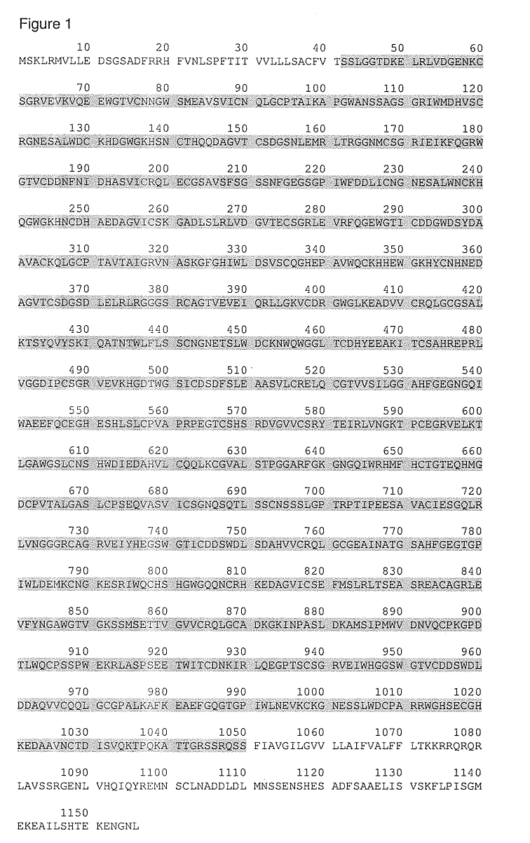

CD163 is a transmembrane haptoglobin-hemoglobin receptor, mainly expressed on macrophages and monocytes, particularly in adipose tissue, and is closely associated with macrophage activation. The amino acid sequence of CD163 is presented in FIG. 1. The extracellular part of CD163 or fragments hereof, may be shed to the blood and is hereby present in a soluble form (sCD163).

All aspects of CD163 measurements herein and all detection methods refer to any form of CD163, membrane-bound or soluble or both. In a preferred embodiment, the measured CD163 is sCD163.

The function of sCD163 is largely unknown, and there is no data to suggest a direct role of sCD163 in the pathogenesis of type 2 diabetes or fatty liver disease [Moestrup S K, Møller H J: CD163: a regulated hemoglobin scavenger receptor with a role in the anti-inflammatory response; Ann Med 2004 36 347-54]. However, levels of sCD163 have previously been reported to be increased in various diseases with enhanced load of monocytes/macrophages and inflammatory components, as rheumatoid arthrititis, Gaucher's disease, liver diseases, and coronary heart disease [Moestrup S K, Møller H J: CD163: a regulated hemoglobin scavenger receptor with a role in the anti-inflammatory response; Ann Med 2004 36 347-54; Aristoteli L P et al: The monocytic lineage specific soluble CD163 is a plasma marker of coronary atherosclerosis; Atherosclerosis 2006 184 342-7; Møller H J et al: Soluble CD163 from activated macrophages predicts mortality in acute liver failure; J Hepatol 2007 47 671-6].

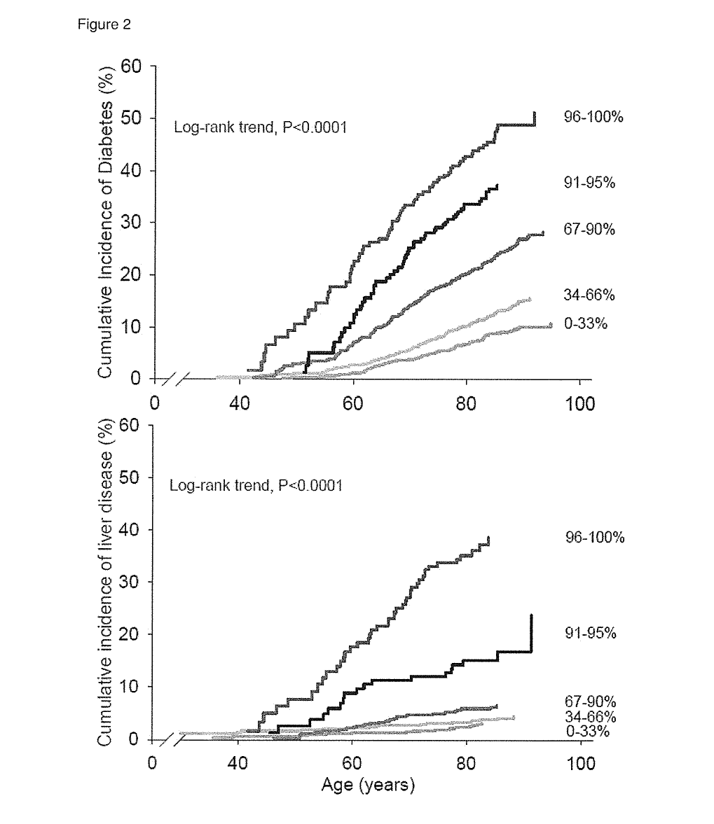

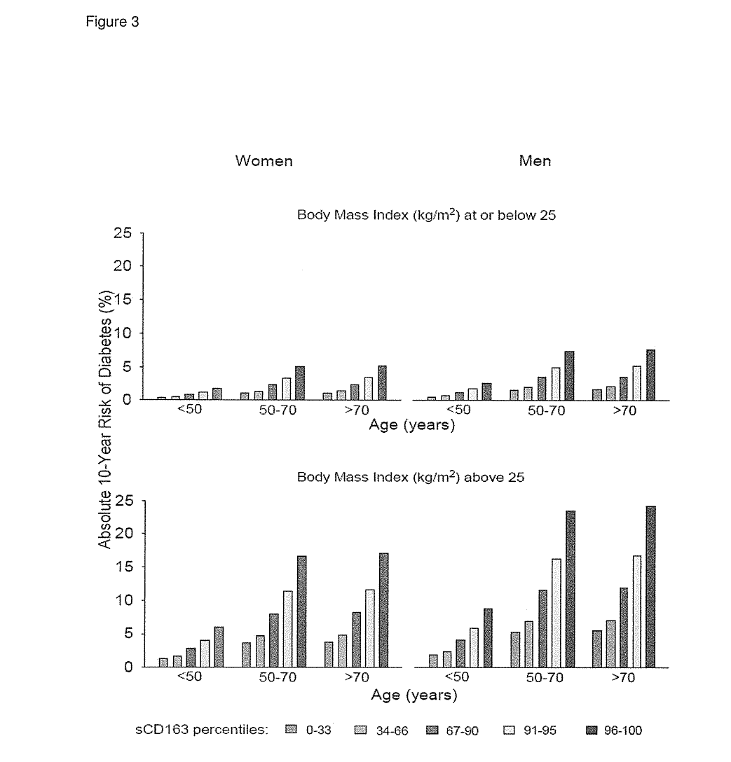

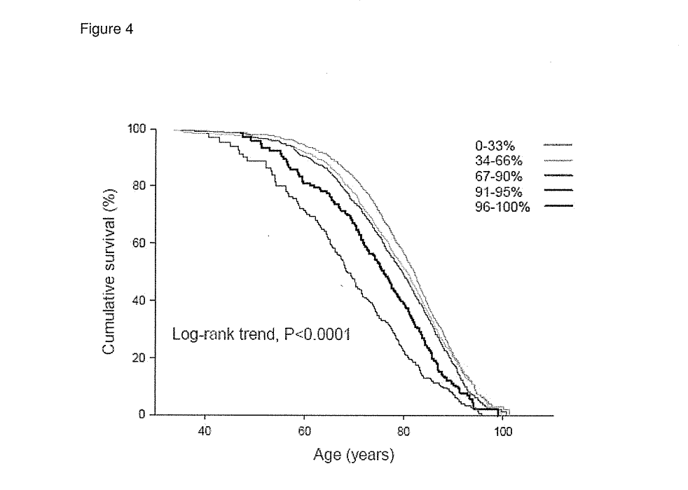

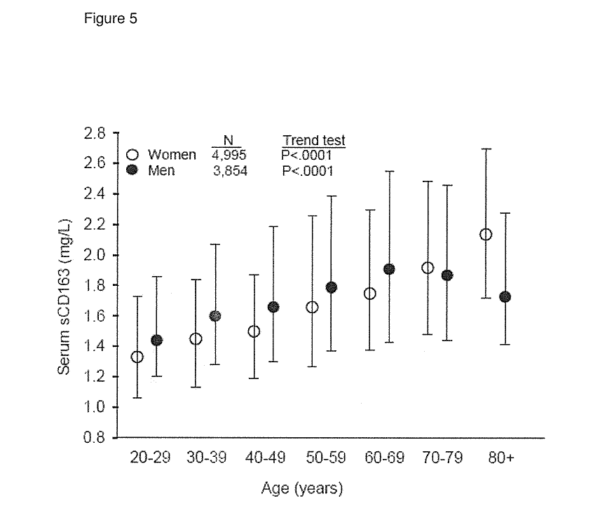

Based on a large Danish investigation involving 8.849 subjects followed for 16 years and monitored for type 2 diabetes and fatty liver disease, the risk of said subjects contracting said diseases can be calculated according to initial blood sCD163 levels and age. Based on plasma sCD163, age and sex, subjects may be divided into five percentile categories: 0-33%, 34-66%, 67-90%, 91-95% and 96-100%, where the lowest percentile relates to subjects with the lowest risk of contracting said diseases, and the highest percentile relates to subjects with the highest risk of contracting said diseases. FIG. 2 shows the cumulative incidence of contracting diabetes and liver disease as a function of age by blood sCD163 levels in the general population.

In a preferred embodiment, said percentiles may be transformed into absolute values (see Table 1, calculated for diabetes 2), where cut-off values are set for the individual age groups, as determined by sCD163 levels in the three highest percentile groups (67-100%).

TABLE 1

| | | | Cut-off | |
|---|---|---|---|---|
| | Cut-off | Number of women | men | Number of men |
| | women | (percentile groups | sCD163 | (percentile groups |
| Age | sCD163 mg/L | 67-100%) | mg/L | 67-100%) |
| 20-29 | 1.58 | 76 | 1.71 | 61 |
| 30-39 | 1.70 | 165 | 1.92 | 153 |
| 40-49 | 1.71 | 191 | 1.93 | 178 |
| 50-59 | 1.98 | 327 | 2.14 | 288 |
| 60-69 | 2.07 | 433 | 2.26 | 310 |
| 70-79 | 2.23 | 404 | 2.24 | 254 |
| 80+ | 2.45 | 94 | 2.04 | 62 |

The three highest percentile groups correspond to 33% of the subjects (67-100%). That is, 33% of the examined subjects have sCD163 values that predicts a 3.4-7.9 fold increased risk of contracting diabetes 2 (2.3-5.0 fold, adjusted multifactorially, see Table 4) as compared to subjects comprising the 33% in the lowest percentile group (0-33%).

Medical Conditions Associated with CD163

The present invention relates to the finding that sCD163 may be used as a prognostic marker for obesity-associated disorders. Based on the investigation of 8849 Danish subjects serum concentrations of sCD163 may be used as an indicator for the risk contracting said disorders, as presented in Table 1 and FIGS. 2 and 3.

Therefore, in a preferred embodiment, the invention relates to the use of CD163 as a prognostic marker where said disorder is low-grade inflammation. In a more preferred embodiment invention relates to the use of CD163 as a prognostic marker where said disorder is diabetes. In a yet more preferred embodiment the invention relates to the use of CD163 as a prognostic marker where said disorder is diabetes 2.

In another preferred embodiment the invention relates to the use of CD163 as a prognostic marker where said disorder is a liver disorder. In a more preferred embodiment, the liver disorder is alcoholic liver disease, such as alcoholic fatty liver, for example alcoholic hepatitis, such as alcoholic fibrosis and sclerosis of liver, for example alcoholic cirrhosis of liver, such as alcoholic hepatic failure (acute, chronic, subacute, with or without hepatic coma).

In another preferred embodiment, the liver disorder is toxic liver disease, such as toxic liver disease with cholestatsis (cholestasis with hepatocyte injury, pure cholestasis), for example toxic liver disease with hepatic necrosis (acute hepatic failure, chronic hepatic failure due to drug abuse), such as toxic liver disease with acute or chronic persistent hepatitis, for example toxic liver disease with chronic lobular hepatitis, such as toxic liver disease with chronic active hepatitis, for example toxic liver disease with lupoid hepatitis, such as toxic liver disease with hepatitis, for example toxic liver disease with fibrosis and cirrhosis of liver, such as toxic liver disease with other disorders of liver (focal nodular hyperplasia, hepatic granulomas, peliosis hepatis, veno-occlusive disease of liver).

In yet another preferred embodiment, the liver disorder is hepatic failure (coma NOS, encephalopathy NOS, acute hepatitis, fulminant hepatitis, malignant hepatitis, liver cell necrosis with hepatic failure), such as acute and subacute hepatic failure, for example chronic hepatic failure.

In yet another preferred embodiment, the liver disorder is chronic hepatitis, not elsewhere classified (NEC), such as chronic persistent hepatitis NEC, for example chronic lobular hepatitis NEC, such as chronic active hepatitis (lupoid hepatitis) NEC, for example other chronic hepatitis NEC.

In yet another preferred embodiment, the liver disorder is fibrosis and cirrhosis of liver, such as hepatic fibrosis, for example hepatic sclerosis, such as hepatic fibrosis with hepatic sclerosis, for example primary biliary cirrhosis (chronic nonsuppurative destructive cholangitis), such as secondary biliary cirrhosis, for example unspecified biliary cirrhosis, such as other and unspecified cirrhosis of liver, for example cryptogenic, macronodular, mixed type, portal or postnecrotic cirrhosis of liver.

In yet another preferred embodiment, the liver disorder is specified as other inflammatory liver diseases such as abscess of liver (cholangitic, haematogenic, lymphogenic or pylephlebtic hepatic abscess), for example phlebitis (pylephlebitis) of portal vein, such as nonspecific reactive hepatitis, for example granulomatus hepatitis NEC, such as autoimmune hepatitis.

In yet another preferred embodiment, the liver disorder is specified as other diseases of liver, such as fatty liver NEC, chronic passive congestion of liver (cirrhosis and sclerosis of liver), for example central haemorrhagic necrosis of liver, such as infarction of liver, for example peliosis hepatitis (hepatic angiomatosis), such as hepatic veno-occlusive disease, for example portal hypertension, such as hepatorenal syndrome, for example other specified diseases of liver, including focal nodular hyperplasia of liver and hepatoptosis.

In yet another preferred embodiment, the liver disorder is classified as liver disorders in other diseases, such as cytomegaloviral, herpesviral or toxoplasma hepatitis, for example hepatosplenic schistosomiasis, such as portal hypertension in schistosomiasis, for example syphilitic liver disease, such as hepatic granulomas in berylliosis and sarcoidosis.

As the level of CD163 may predict the risk of contracting said disorders, and contracting said disorders are associated with reduced life expectancy, the level of CD163 may therefore, in a preferred embodiment, be used as a prognostic marker for a disorder where said disorder is reduced life expectancy. In a more preferred embodiment, said risk is a risk of contracting said disorders within a time frame of 1-20 years, such as in the range of 1-2 years, for example 2-5 years, such as 5-7 years, for example 7-10 years, such as 10-15 years, for example 15-20 years.

### Sampling of CD163

The present invention relates to the use of CD163 as a prognostic marker for the assessment of the risk for contracting a disorder. In a preferred embodiment, the level of CD163 will be obtained from a biological sample, such as serum, for example plasma, such as whole blood, for example saliva, such as urine, for example lymph, such as a biopsy, for example semen, such as faeces, for example tears, such as sweat, for example milk, such as cerebrospinal fluid, for example ascites fluid, such as for example synovial fluid. Preferably the sample is blood, plasma or serum. More preferably the sample is plasma or serum.

### Methods for Determining CD163

Point of Care test preferably relies on a lateral flow test based on an immunological principle. Lateral flow tests are also known as lateral flow immunochromatographic assays and are simple devices intended to detect the presence (or absence) of a target analyte in sample. Often produced in a dipstick format, a lateral flow test is a form of immunoassay in which the test sample flows along a solid substrate, preferably via capillary action. After the sample is applied to the test it preferably encounters a coloured reagent which mixes with the sample and transits the substrate encountering lines or zones which have been pretreated with an antibody or antigen. Depending upon the analytes present in the sample the coloured reagent can become bound at the test line or zone. Semi-quantitative lateral flow tests can operate as either competitive or sandwich assays:

In a preferred embodiment, the sample is mixed with CD163 antibody-coated microparticles with a resulting change in the turbidity of the sample. The turbidity change may then be correlated with the amount of CD163 in the sample when compared with a reference sample.

In another preferred embodiment, the level of CD163 is detected by nephelometry where an antibody and the antigen are mixed in concentrations such that only small aggregates are formed. These aggregates will scatter light (usually a laser) passed through it rather than simply absorbing it. The fraction of scattered light is determined by collecting the light at an angle where it is measured and compared to the fraction of scattered light from known mixtures. Scattered light from the sample is determined by using a standard curve.

In another preferred embodiment, the sample moves from the application site where it, for example, is mixed with antibody-coated nanoparticles in lateral flow/diffusion through a (e.g. nitrocellulose-) membrane. At one point on the way another CD163 antibody is fixed in the membrane making the CD163-primary antibody complex to halt. The nano-particle (preferably colloidal gold/dyed latex) will give a visual line.

In another embodiment, the sample is applied through a (e.g. nitrocellulose-) membrane coated with a primary CD163 antibody. The sample CD163 is then recognised and bound by the primary CD163 antibody. The immobilised CD163 on the membrane may then be recognised by (preferably colloidal gold/dyed latex) particles conjugated with another CD163 antibody, and the complex will develop a colour reaction, which intensity corresponds to the amount of CD163 in the sample.

For large-scale detection and more precise quantitative measurement of CD163 in a sample, several methods may be applied:

In another preferred embodiment, the level of CD163 is detected by radioimmunoassay (RIA). RIA is a very sensitive technique used to measure concentrations of antigens without the need to use a bioassay. To perform a radioimmunoassay, a known quantity of an antigen is made radioactive, frequently by labeling it with gamma-radioactive isotopes of iodine attached to tyrosine. This radio labeled antigen is then mixed with a known amount of antibody for that antigen, and as a result, the two chemically bind to one another. Then, a sample of serum from a patient containing an unknown quantity of that same antigen is added. This causes the unlabeled (or “cold”) antigen from the serum to compete with the radio labeled antigen for antibody binding sites. As the concentration of “cold” antigen is increased, more of it binds to the antibody, displacing the radio labeled variant, and reducing the ratio of antibody-bound radio labeled antigen to free radio labeled antigen. The bound antigens are then separated from the unbound ones, and the radioactivity of the free antigen remaining in the supernatant is measured. Using known standards, a binding curve can then be generated which allows the amount of antigen in the patient's serum to be derived. In this assay, the binding between antibody and antigen may be substituted by any protein-protein or protein-peptide interaction, such as ligand-receptor interaction, for example CD163-haemoglobin or CD163-haemoglobin/haptoglobin binding.

In a preferred embodiment, the level of CD163 is detected by enzyme-linked immunosorbent assay (ELISA). ELISA is a quantitative technique used to detect the presence of protein, or any other antigen, in a sample. In ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal.

Several types of ELISA exist:

### Indirect ELISA

### Sandwich ELISA

### Competitive ELISA

### Reverse ELISA

Other immuno-based assays may also be used to detect CD163 in a sample, such as chemiluminescent immunometric assays and Dissociation-Enhanced Lanthinide Immunoassays.

In a preferred embodiment, the level of CD163 is detected by chromatography-based methods, more specifically liquid chromatography. Therefore, in a more preferred embodiment, the level of CD163 is detected by affinity chromatography which is based on selective non-covalent interaction between an analyte and specific molecules.

In another preferred embodiment, the level of CD163 is detected by ion exchange chromatography which uses ion exchange mechanisms to separate analytes. Ion exchange chromatography uses a charged stationary phase to separate charged compounds. In conventional methods the stationary phase is an ion exchange resin that carries charged functional groups which interact with oppositely charged groups of the compound to be retained.

In yet another preferred embodiment, the level of CD163 is detected by size exclusion chromatography (SEC) which is also known as gel permeation chromatography (GPC) or gel filtration chromatography. SEC is used to separate molecules according to their size (or more accurately according to their hydrodynamic diameter or hydrodynamic volume). Smaller molecules are able to enter the pores of the media and, therefore, take longer to elute, whereas larger molecules are excluded from the pores and elute faster.

In yet another preferred embodiment, the level of CD163 is detected by reversed-phase chromatography which is an elution procedure in which the mobile phase is significantly more polar than the stationary phase. Hence, polar compounds are eluted first while non-polar compounds are retained.

In a preferred embodiment, the level of CD163 is detected by electrophoresis. Electrophoresis utilizes the motion of dispersed particles relative to a fluid under the influence of an electric field. Particles then move with a speed according to their relative charge. More specifically, the following electrophoretic methods may be used for detection of CD163:

Sodium dodecyl sulfate polyacrylamide gel electrophoresis (SDS-PAGE).
Rocket immunoelectrophoresis.
Affinity immunoelectrophoresis.
Isoelectric focusing.

In a preferred embodiment, the level of CD163 is detected by flow cytometry. In flow cytometry a beam of light of a single wavelength is directed onto a hydrodynamically-focused stream of fluid. A number of detectors (some fluorescent) are aimed at the point where the stream passes through the light beam: one in line with the light beam and several detectors perpendicular to it. Each suspended particle from 0.2 to 150 micrometers passing through the beam scatters the light in some way, and fluorescent chemicals found in the particle or attached to the particle may be excited into emitting light at a longer wavelength than the light source. This combination of scattered and fluorescent light is picked up by the detectors, and, by analysing fluctuations in brightness at each detector, it is then possible to derive various types of information about the physical and chemical structure of each individual particle.

In a preferred embodiment, the level of CD163 is detected by Luminex technology, which is based on a technique where microspheres are coated with reagents specific to capture a specific antigen from a sample.

In a preferred embodiment, the level of CD163 is detected by mass spectrometry (MS). MS is an analytical technique for the determination of the elemental composition of a sample or molecule. It is also used for elucidating the chemical structures of molecules, such as proteins and other chemical compounds. The MS principle consists of ionizing chemical compounds to generate charged molecules or molecule fragments and measurement of their mass-to-charge ratios.

### Population Groups at Risk

Based on an investigation involving 8.849 subjects followed for 16 years and monitored for type 2 diabetes and fatty liver disease, the risk of said subjects contracting said diseases may be predicted according to CD163 levels and age. By determining CD163 levels, age and sex, subjects may be divided into five percentile categories: 0-33%, 34-66%, 67-90%, 91-95% and 96-100%, where the lowest percentile relates to subjects with the lowest risk of contracting said diseases, and the highest percentile relates to subjects with the highest risk of contracting said diseases. Prospective reduced life expectancy may also be predicted based on CD163 levels.

Therefore, in a preferred embodiment, the size of the population preferably needed for calculating said risk of contracting said diseases by determining the amount of CD163 is within the range of 100-10.000 people, such as between 100-500 people, for example 500-1.000 people, such as 1.000-2.000 people, for example 2.000-2.500 people, such as 2.500-5.000 people, for example 5.000-7.500 people, such as for example in the range of 7.500-10.000 people.

In another preferred embodiment, an individual of said population is judged to have a high CD163 level when a high CD163 level comprises a value found in individuals belonging to a percentile with a lower limit of at least 60%, more preferably at least 65%, more preferable at least 67%, more preferable at least 70%, more preferably at least 75%, more preferably at least 80%, more preferably at least 85%, more preferably at least 90%, more preferably at least 95%, more preferably at least 97%, more preferably with a lower limit of at least 100%.

In another preferred embodiment, said percentiles are determined for a subset of individuals, said individuals having the same gender or race, or belonging to a group based on age, BMI, smoking habit, occupation, physical inactivity, hip circumference, waist circumference, systolic and/or diastolic blood pressure, alcohol consumption, a combination of any subset of these, or other risk factor. In a more preferred embodiment, said percentiles are determined for a subset of individuals, said individuals having the same gender and belonging to the same age interval, said interval being 5 years, 10 years, 15 years, 20 years or said interval being 25 years.

Said percentiles are based on multiple factors, among those CD163 levels, gender and age. When classified into 10-year age intervals, it is possible to derive absolute cut-off values, above which an individual is at risk of contracting said disorders.

In yet another preferred embodiment, a high level of CD163 for said individuals is determined according to Table 1. More specifically, in a preferred embodiment wherein said individual is a female of at least 20 years, a high level of sCD163 is at least 1.58 mg/L serum, or wherein said individual is a female of at least 30 years, a high level of sCD163 is at least 1.7 mg/L serum, or wherein said individual is a female of at least 40 years, a high level of sCD163 is at least 1.71 mg/L serum, or wherein said individual is a female of at least 50 years, a high level of sCD163 is at least 1.98 mg/L serum, or wherein said individual is a female of at least 60 years, a high level of sCD163 is at least 2.07 mg/L serum, or wherein said individual is a female of at least 70 years, a high level of sCD163 is at least 2.23 mg/L serum, or wherein said individual is a female of at least 80 years, a high level of sCD163 is at least 2.45 mg/L serum, or wherein said individual is a male of at least 20 years, a high level of sCD163 is at least 1.71 mg/L serum, or wherein said individual is a male of at least 30 years, a high level of sCD163 is at least 1.92 mg/L serum, or wherein said individual is a male of at least 40 years, a high level of sCD163 is at least 1.93 mg/L serum, or wherein said individual is a male of at least 50 years, a high level of sCD163 is at least 2.14 mg/L serum, or wherein said individual is a male of at least 60 years, a high level of sCD163 is at least 2.26 mg/L serum, or wherein said individual is a male of at least 70 years, a high level of sCD163 is at least 2.24 mg/L serum, or wherein said individual is a male of at least 80 years, a high level of sCD163 is at least 2.04 mg/L serum.

The risk of contracting diabetes among said individuals may be determined from which percentile an individual belongs to. The risk of contracting said disease is calculated by comparing to a reference group.

In a preferred embodiment, the risk of said individual contracting diabetes over a time period of 20 years is preferably at least 2 times as high as for the reference group, more preferably at least 5 times as high, most preferably at least 8 times as high as for the reference group. In another preferred embodiment, the time period in which the risk of said individual contracting said disease is higher as for the reference group is preferably 15 years, such as 10 years, for example 5 years. In another preferred embodiment, the reference group is the age and/or gender group to which said individual belongs with the age group being 5 years, such as 10 years, for example 15 years, such as 20 years, the age group being for example 25 years. In a yet more preferred embodiment, the reference group constituting the lowest percentile group for CD163, being 0-33%.

The risk of contracting liver disease among said individuals may be determined from which percentile an individual belongs to. The risk of contracting said disease is calculated by comparing to a reference group.

In a preferred embodiment, the risk of said individual contracting liver disease over a time period of 20 years is preferably at least 2 times as high as for the reference group, more preferably at least 5 times as high, more preferably at least 10 times as high, even more preferably at least 15 times as high, such as at least 20 times as high, most preferably 25 times as high as for the reference group. In another preferred embodiment, the time period in which the risk of said individual contracting said disease is higher as for the reference group is preferably 15 years, such as 10 years, for example 5 years. In another preferred embodiment, the reference group is the age and/or gender group to which said individual belongs with the age group being 5 years, such as 10 years, for example 15 years, such as 20 years, the age group being for example 25 years. In a yet more preferred embodiment, the reference group constituting the lowest percentile group for CD163, being 0-33%.

Divided into percentiles based on CD163 levels, age and gender, a preferred reference group is the group with the lowest risk of contracting diabetes or a liver disease and without risk of reduced life expectancy.

In a preferred embodiment, a subject at high risk according to said parameters preferably has a reduced life expectancy of at least 2 years shorter than the average of the reference group of individuals, for example at least 5 years shorter, such as at least 10 years shorter, for example 15 years shorter than the average of the reference group of individuals. In another preferred embodiment, the reference group is the age and/or gender group to which said individual belongs with the age group being 5 years, such as 10 years, for example 15 years, such as 20 years, the age group being for example 25 years. In a yet more preferred embodiment, the reference group constituting the lowest percentile group for CD163, being 0-33%.

### Additional Assessments

In the investigation that the present invention is based upon, samples were collected from 8.849 individuals of Danish descent. Approximately 99% of these individuals were Caucasian.

In a preferred embodiment, the present invention relates to the use of CD163 as a prognostic marker for contracting diabetes 2, a liver disorder or for an individual to have a reduced life expectancy when said individual is Caucasian. The data are likely to be valid for non-Caucasians as well. The principle of dividing a group of individuals into percentile groups, e.g., according to the risk of contracting a disease or based on other parameters determining any risk, may apply to any race, population group, or other groups of individuals. Therefore, if supporting clinical and/or biochemical data are present, the use of sCD163 may be used as a prognostic marker in any population group. Thus, an individual of any race belonging to a given CD163 percentile is expected to have the same risk of contracting a disorder as Caucasians belonging to the same percentile group.

Several biochemical parameters are known to be associated with obesity-related diseases. A normal procedure in the clinical laboratory may be to confirm positive and negative findings obtained by assessing one biochemical marker (of for example a disorder) by assessing the presence of other, independent biochemical markers with similar clinical indications.

In another preferred embodiment the use of CD163 as a prognostic marker for said diseases may be supported by assessing measures such as BMI, smoking habits, occupation, physical inactivity, hip circumference, waist circumference, systolic and/or diastolic blood pressure, alcohol consumption or other, related biochemical markers obtained from a group of, but not limited to, blood glucose, cholesterol (LDL, HDL and/or total), triglycerides, apolipoprotein, CRP, Fibrinogen, alphal-antitrypsin, ALAT, gammaGT, alkaline phosphatise, lactate dehydrogenase, homocysteine, and bilirubine.

Treatment of Subjects with Increased CD163

One great asset of a prognostic marker is that it paves the way for an individual to take actions aimed at preventing a certain disease to develop before overt signs of said disease develop. In the case of the present invention, which relates to the detection of an elevated level of CD163, said actions may include altered daily routines, such as increased physical activity and a healthier diet, such as reduced consumption of fat, sugar and alcohol. Moreover, a number of compounds are undergoing clinical trials to investigate their effect on lowering low-grade systemic inflammation or subclinical inflammation. Examples of such drugs include but are not limited to:

Coffee, Glucose-dependent insulinotropic polypeptide (GIP), nicotinic acid, pioglitazone, ramipril, curcumin, fructanes, acarbose, vitamin D, butyrate, thiazolidinediones, mesalazine, salsalate, advair, flovent, atenolol, ramipril, metformin and resveratrol.

Metformin (N,N-dimethylimidodicarbonimidic diamide) is an oral anti-diabetic drug from the biguanide class that originates from the French lilac (Galega officinalis) plant. The main use for metformin is in the treatment of diabetes 2, especially when this accompanies obesity and insulin resistance.

Resveratrol (3,5,4′-trihydroxystilbene) is a polyphenolic phytoalexin. It is a stilbenoid, a derivate of stilbene, and is produced in plants with the help of the enzyme stilbene synthase. It exists as two structural isomers: cis-(Z) and trans-(E), with the trans-isomer shown in the top image. The trans-form can undergo isomerisation to the cis-form when heated or exposed to ultraviolet irradiation. Resveratrol is a polyphenol found in red wine.

### Statins.

The statins (or HMG-CoA reductase inhibitors) are a class of drugs that lower cholesterol levels in people with or at risk of cardiovascular disease. They lower cholesterol by inhibiting the enzyme HMG-CoA reductase, which is the rate-limiting enzyme of the mevalonate pathway of cholesterol synthesis. Inhibition of this enzyme in the liver results in decreased cholesterol synthesis as well as increased synthesis of LDL receptors, resulting in an increased clearance of low-density lipoprotein (LDL) from the bloodstream. The statin family presently includes:

| Statin | Brand name | Derivation |
|---|---|---|
| Atorvastatin | Lipitor, Torvast | Synthetic |
| Cerivastatin | Lipobay, Baycol. (Withdrawn from | Synthetic |
| | the market in August, 2001 due to | |
| | risk of serious Rhabdomyolysis) | |
| Fluvastatin | Lescol, Lescol XL | Synthetic |
| Lovastatin | Mevacor, Altocor, Altoprev | Fermentation-derived. Naturally- |
| | | occurring compound. Found in |
| | | oyster mushrooms and red yeast |
| | | rice |
| Mevastatin | — | Naturally-occurring compound. |
| | | Found in red yeast rice |
| Pitavastatin | Livalo, Pitava | Synthetic |
| Pravastatin | Pravachol, Selektine, Lipostat | Fermentation-derived |
| Rosuvastatin | Crestor | Synthetic |
| Simvastatin | Zocor, Lipex | Fermentation-derived. (Simvastatin |
| | | is a synthetic derivate of a |
| | | fermentation product) |
| Simvastatin + | Vytorin | Combination therapy |
| Ezetimibe | | |
| Lovastatin + | Advicor | Combination therapy |
| Niacin | | |
| extended- | | |
| release | | |
| Atorvastatin + | Caduet | Combination therapy— |
| Amlodipine | | |
| Besylate | | Cholesterol + Blood Pressure |
| Simvastatin + | Simcor | Combination therapy |
| Niacin | | |
| extended- | | |
| release | | |

### EXAMPLES

### Example 1

### The Level of sCD163 Predicts Risk of Diabetes, a Liver Disorder and Reduced Life Expectancy in the General Population

### Abstract

The incidence of obesity and associated diseases such as type 2 diabetes and fatty liver disease has increased drastically during recent decades and now constitutes a serious health threat globally. The inventors tested whether a new biomarker, sCD163, identifies at-risk individuals before overt disease has developed.

### Materials and Methods

### Study Participants

The inventors used a population-based prospective study of the Danish general population, the 1991 to 1994 examination of the Copenhagen City Heart Study [Frikke Schmidt R, et al: Association of Loss-of-Function Mutations in the ABCA1 Gene With High-Density Lipoprotein Cholesterol Levels and Risk of Ischemic Heart Disease; JAMA 2008 299 2524-32; Schnohr P et al: The Copenhagen City Heart Study, Østerbroundersøgelsen, tables with data from the third examination 1991-1994; Eur Heart J 2001 3(Supplement H) 1-83]. Participants age 20 years and older were selected randomly after sex and age stratification into 5-year groups among residents of Copenhagen. Of the 17,180 subjects invited, 10,135 participated, and plasma was available for sCD163 determination in 8,849 participants. Participants were followed using their unique Central Person Register number from baseline at the 1991 to 1994 examination until July 2007. Follow-up was 100% complete. Roughly 99% were Caucasians of Danish descent. The participants filled out a self-administered questionnaire, which was validated by the participant and an investigator on the day of attendance. Participants reported on smoking and physical activity habits and on alcohol consumption. Body mass index was measured weight in kilograms divided by measured height in meters squared. Waist in cm, hip in cm and blood pressure in mmHg were measured. Plasma sCD163 was measured a second time in blood samples of 923 participants of the 2001-2003 examination of the Copenhagen City Heart Study cohort. These participants were free of known diseases at the 1991-1994 and 2001-2003 examinations, allowing correction for regression dilution bias [Clarke R et al: Underestimation of Risk Associations Due to Regression Dilution in Long-term Follow-up of Prospective Studies; Am J Epidemiol 1999 150 341-53].

### End Points

Information on diagnoses of type 2 diabetes (World Health Organization; International Classification of Diseases (ICD), 8^{th} edition: code 250; 10^{th} edition: codes E10-E14) and all benign non-malignant, non-infectious liver disease, representing fatty liver disease (ICD8: codes 570, 5710, 5711, 5719, and 5730; ICD10: codes K70-K74, and K76K), was collected from the national Danish Patient Registry and the national Danish Causes of Death Registry.

### Ethics

Studies were approved by institutional review boards and Danish ethical committees (KF V.100.2039/91 and KF 01-144/01), Copenhagen and Frederiksberg committee, and conducted according to the Declaration of Helsinki. Written informed consent was obtained from participants.

Laboratory analysis

Plasma levels of sCD163 were determined in samples frozen for 12 to 15 years at −80° C. by a sandwich enzyme-linked immunosorbent assay as previously described [Møller H J et al: Characterization of an enzyme-linked immunosorbent assay for soluble CD163; Scand J Clin lab Invest 2002 62 293-9]. The recovery of the enzyme-linked immunosorbent assay was 106% and the minimum detection limit was below 6.25 μg/L. Glucose levels were measured by a standard hexokinase/G6P-DH assay in plasma [Schnohr P et al: The Copenhagen City Heart Study, Østerbroundersøgelsen, tables with data from the third examination 1991-1994; Eur Heart J 2001 3(Supplement H) 1-83]. High-sensitivity C Reactive Protein (CRP), fibrinogen, alfa1-antitrypsin and orosomucoid were measured by standard nephelometry or turbidimi hospital assays. Colorimetric and turbidimetric assays were used to measure plasma levels of total cholesterol, triglycerides, HDL cholesterol after precipitation of apolipoprotein B containing lipoproteins, apolipoproteins B and -AI (all Boehringer Mannheim GmbH, Mannheim, Germany). Low-density lipoprotein (LDL) cholesterol was calculated according to Friedewald if triglycerides were <354 mg/dL (4 mmol/L) [Friedewald WT et al: Estimation of the concentration of low-density lipoprotein cholesterol in plasma, without use of the preparative ultracentrifuge; Clin Chem 1972 18 499-502], but measured directly at higher triglyceride levels (Konelab, Helsinki, Finland).

### Statistical Analysis

The inventors used STATA version 10 (Stata Corp LP, College Station, Tex.). Two-sided P<0.05 was considered significant. Kruskal-Wallis analysis of variation nonparametric trend test, Mann-Whitney U test, and Spearman's rho correlation were used. Plasma sCD163 levels were stratified into categories according to plasma sCD163 percentiles in sex and 10-year age groups: five percentile categories were 1% to 33%, 34% to 66%, 67% to 90%, 91% to 95%, and 96% to 100%. The five percentile groups were prespecified as for previous tests of biomarkers from the same study population [Kamstrup P R et al: Extreme Lipoprotein(a) Levels and Risk of Myocardial Infarction in the General Population: The Copenhagen City Heart Study; Circulation 2008 117 176-84; Johansen J S et al: Elevated Plasma YKL-40 Predicts Increased Risk of Gastrointestinal Cancer and Decreased Survival After Any Cancer Diagnosis in the General Population; J Clin Oncol 2009 27 572-8] in order to evaluate both tertiles in the lower range and extreme phenotypes in the upper range. Cumulative incidence was plotted using Kaplan-Meier curves, and differences between plasma sCD163 percentile categories examined using log-rank tests. Hazard ratios and 95% CIs were calculated using Cox regression analysis with age as the time scale (left-truncation which implies that age is automatically accounted for) and adjusted for sex or multifactorially (sex, smoking habits, physical activity, body mass index, alcohol consumption, blood pressure). Participants with events before blood sampling (n=155) were excluded from prospective analyses of type 2 diabetes. Hazard ratios were corrected for regression dilution bias using a nonparametric method [Clarke R et al: Underestimation of Risk Associations Due to Regression Dilution in Long-term Follow-up of Prospective Studies; Am J Epidemiol 1999 150 341-53]. For this correction we used plasma sCD163 values from 923 individuals attending both the 1991 to 1994 baseline examination and the 2001 to 2003 follow-up examination; however, the main analyses were conducted on all 8,849 individuals. A regression dilution ratio of 0.86 was computed. Absolute 10-year risk of type 2 diabetes by plasma sCD163 percentile categories was estimated by using the regression coefficients from a poisson regression model including only the most significant covariates from the Cox regression models: sex, age in three groups (<50, 50 to 70, >70 years), and body mass index in two groups (≦25, >25 kg/m²) at the date of blood sampling. Absolute risks are presented as estimated incidence rates (events/10 years) in percentages.

### Results

Median plasma sCD163 was 1.71 mg/L (interquartile range, 1.31 to 2.26 mg/L) in women and 1.76 mg/dL (interquartile range, 1.37 to 2.36 mg/L) in men (P<0.0001). Plasma sCD163 levels increased in both sexes with increasing age (P for trends, <0.0001) (see FIG. 5). Spearman's rho correlation between serum sCD163 and age was 0.22 (P<0.0001, Table 2).

### Baseline Characteristics

Baseline characteristics of participants according to plasma sCD163 percentile categories (grouped by 10-year age and sex) are given in Table 2. Increasing plasma concentrations of sCD163 in age and sex adjusted percentile categories were associated with increasing body mass index, waist, hip, waist hip ratio, systolic blood pressure, and diastolic blood pressure (all P for trends, <0.0001). Spearman's rho correlations between unadjusted plasma sCD163 levels and baseline anthropometric factors were strongest for waist circumference, body mass index, waist hip ratio, hip circumference, and systolic blood pressure (Spearman's rho: 0.30, 0.29, 0.24, 0.22, 0.22, respectively, all P-values <0.0001) (Table 2).

sCD163 and Biochemical Parameters at Baseline

Increasing serum concentrations of sCD163 in age and sex adjusted percentile categories were associated with increasing levels of glucose and inflammatory markers as CRP, fibrinogen, alfa1-antitrypsin, and orosomucoid (all P for trends, <0.0001), as well as associated with LDL cholesterol (P for trend, 0.01), apolipoprotein B (P for trend, 0.02), and HDL cholesterol, apolipoprotein AI, and triglycerides (P for trends, <0.0001) (Table 3). Spearman's rho correlations between unadjusted serum sCD163 levels and baseline biochemical parameters are presented in Table 3, and were strongest for levels of orosomucoid, CRP, triglycerides, and fibrinogen (Spearman's rho: 0.26, 0.25, 0.23, 0.22, respectively, all P-values <0.0001).

sCD163 and Risk of Type 2 Diabetes and Fatty Liver Disease

During 16 years of follow-up, 511 of 8,694 event-free participants developed diabetes. The cumulative incidence of diabetes as a function of age was increased with increasing plasma sCD163 percentile categories (log-rank P for trend, <0.0001) (FIG. 2). At the age of 80 years, 10%, 20%, 34%, and 43%, respectively, of individuals with sCD163 in 34-66%, 67%-90%, 91%-95%, and 96%-100% categories had type 2 diabetes compared with 7% for the 0%-30% category.

Multifactorially adjusted (age, sex, smoking, physical inactivity, body mass index, alcohol consumption, systolic blood pressure and diastolic blood pressure) hazard ratios for diabetes were 1.3 (95% confidence interval (CI), 1.0 to 1.7) for plasma sCD163 percentile category 34% to 66%, 2.1 (95% CI, 1.6 to 2.7) for 67% to 90%, 2.8 (95% CI, 1.9-3.9) for 91% to 95%, and 4.0 (95% CI, 2.9 to 5.6) for 96% to 100% versus plasma sCD163 percentile category 0% to 33% (P for trend, <0.0001) (Table 4, upper part).

During 16 years of follow-up, 136 participants developed fatty liver disease. The cumulative incidence of fatty liver disease as a function of age was increased with increasing plasma sCD163 percentile categories (log-rank P for trend, <0.0001). At the age of 80 years, 3%, 5%, 15%, and 35%, respectively, of individuals with sCD163 in 34-66%, 67%-90%, 91%-95%, and 96%-100% categories had fatty liver disease compared with 2% for the 0%-30% category. HRs for fatty liver disease ranged from 0.9 to 22 as a function of increasing percentile category (Table 4, lover part).

### Absolute 10-Year Risk of Type 2 Diabetes

The lowest absolute 10-year risk for diabetes was 1% in women aged younger than 50 years, with body mass index at or below 25 and in serum sCD163 percentile category 0% to 33%. Absolute risk was generally higher in men than in women and increased with increasing age and body mass index above 25. The highest absolute 10-year risk for diabetes was 17% and 24% in women and men, respectively, age older than 70 years, with body mass index above 25 and in serum sCD163 percentile category 96% to 100% (FIG. 3).

Proportion Surviving as a Function of Age by Plasma sCD163 Levels in the General Population

The proportion surviving decreased with increased sCD163 percentile categories (log-rank P for trend test, <0.0001). The median survival was decreased with 13.5 years in the 96-100% percentile category compared with the 0-33% percentile category. This decrease in lifespan is greater that the lifespan loss observed in smokers (approx. 9 years).

TABLE 2
Baseline characteristics of study participants from the general population.

| | Categories by sex and 10-year age plasma sCD163 percentile | | | | | | | | | | Trend | Spearmans |
|---|---|---|---|---|---|---|---|---|---|---|---|---|
| | 0-33% | | 34-66% | | 67-90% | | 91-95% | | 96-100% | | P-value | rho |
| Number (%) | 2,944 | (33) | 2,909 | (33) | 2,118 | (24) | 443 | (5) | 435 | (5) | — | — |
| Women, No. (%) | 1,655 | (56) | 1,650 | (57) | 1,195 | (56) | 250 | (56) | 245 | (56) | — | — |
| Age (years) | 61 | (48-71) | 61 | (48-71) | 61 | (48-71) | 61 | (49-71) | 61 | (47-71) | — | 0.22* |
| Smoking, No. (%) | 2,277 | (78) | 2,110 | (73) | 1,524 | (72) | 316 | (72) | 332 | (77) | — | — |
| Physical inactivity, | 1,870 | (64) | 1,869 | (65) | 1,424 | (68) | 311 | (71) | 308 | (72) | — | — |
| No. (%) | | | | | | | | | | | | |
| Body mass index | 24 | (22-27) | 25 | (22-28) | 26 | (23-29) | 27 | (24-31) | 26 | (23-30) | <0.001 | 0.29* |
| (kg/m²) | | | | | | | | | | | | |
| Waist (cm) | 84 | (75-93) | 86 | (77-96) | 90 | (80-100) | 94 | (84-105) | 93 | (84-102) | <0.001 | 0.30* |
| Hip (cm) | 98 | (93-103) | 99 | (94-105) | 101 | (96-106) | 102 | (96-110) | 101 | (94-107) | <0.001 | 0.22* |
| Waist hip ratio | 0.86 | (0.79-0.92) | 0.87 | (0.80-0.94) | 0.89 | (0.82-0.96) | 0.91 | (0.84-0.98) | 0.91 | (0.84-0.99) | <0.001 | 0.24* |
| Alcohol | 10 | (3-21) | 9 | (2-21) | 9 | (2-22) | 9 | (0-24) | 12 | (2-34) | 0.95 | −0.01 |
| consumption (g/day) | | | | | | | | | | | | |
| Systolic blood | 135 | (121-150) | 136 | (122-153) | 139 | (125-156) | 140 | (125-157) | 140 | (125-156) | <0.001 | 0.22* |
| pressure (mmHg) | | | | | | | | | | | | |
| Diastolic blood | 83 | (75-90) | 84 | (76-92) | 85 | (77-94) | 86 | (78-94) | 85 | (77-95) | <0.001 | 0.15* |
| pressure (mmHg) | | | | | | | | | | | | |

Values are expressed as numbers, percent, or median (interquartile range).
Statistical comparisons between the five sCD163 percentile categories were made using trend test.
Spearmans rho was calculated on unadjusted plasma sCD163.
*P < 0.00001.
Smoking: Current or exsmokers at baseline.
Physical inactivity: Individuals with less than 2 to 4 hours per week of light physical activity at baseline.

TABLE 3
Relation between sCD163 and biochemical parameters at baseline in the general population.

| | Categories by sex and 10-year age plasma sCD163 percentile | | | | | Trend | Spearmans |
|---|---|---|---|---|---|---|---|
| | 0-33% | 34-66% | 67-90% | 91-95% | 96-100% | P-value | Rho |
| Glucose homeostasis | | | | | | | |
| Glucose (mmol/L) | 5.4 (5.0-5.9) | 5.4 (5.0-6.1) | 5.5 (5.0-6.2) | 5.7 (5.1-6.8) | 5.7 (5.1-7.0) | <0.001 | 0.17* |
| Inflammatory markers | | | | | | | |
| CRP (mg/L) | 1.54 (1.19-2.45) | 1.67 (1.23-2.81) | 1.98 (1.36-3.43) | 2.46 (1.56-4.78) | 2.52 (1.48-5.02) | <0.001 | 0.25* |
| Fibrinogen (g/L) | 2.86 (2.39-3.39) | 2.99 (2.50-3.58) | 3.11 (2.59-3.72) | 3.16 (2.63-3.81) | 3.14 (2.49-3.88) | <0.001 | 0.22* |
| Alfa1-antitrypsin (μmol/L) | 24.9 (22.3-28.0) | 25.0 (22.3-28.1) | 25.3 (22.4-28.1) | 25.6 (22.8-28.9) | 26.7 (23.8-30.2) | <0.001 | 0.08* |
| Orosomucoid (μmol/L) | 19.5 (17.0-23.4) | 21.4 (18.3-25.3) | 22.4 (19.5-26.3) | 23.4 (19.5-27.3) | 23.4 (18.5-30.2) | <0.001 | 0.26* |
| Lipid traits | | | | | | | |
| Total Cholesterol (mmol/L) | 6.0 (5.2-6.9) | 6.1 (5.3-7.0) | 6.1 (5.3-7.1) | 6.0 (5.2-6.9) | 5.8 (4.9-6.7) | 0.46 | 0.10* |
| LDL Cholesterol (mmol/L) | 3.6 (3.0-4.4) | 3.7 (3.0-4.5) | 3.7 (3.0-4.5) | 3.6 (2.9-4.4) | 3.4 (2.6-4.1) | 0.01 | 0.07 |
| Apolipoprotein B (mg/dL) | 83 (70-99) | 86 (70-101) | 87 (71-103) | 87 (73-103) | 81 (67-99) | 0.02 | 0.12* |
| HDL Cholesterol (mmol/L) | 1.6 (1.3-1.9) | 1.5 (1.2-1.9) | 1.4 (1.2-1.8) | 1.3 (1.0-1.7) | 1.4 (1.1-1.7) | <0.001 | −0.17* |
| Apolipoprotein Al (mg/dL) | 143 (124-164) | 139 (122-160) | 136 (120-155) | 130 (113-154) | 135 (115-154) | <0.001 | −0.11* |
| Triglycerides (mmol/L) | 1.40 (1.03-1.96) | 1.54 (1.08-2.21) | 1.64 (1.17-2.44) | 1.92 (1.32-2.84) | 1.88 (1.20-2.71) | <0.001 | 0.23* |
| Liver parametres | | | | | | | |
| ALAT (U/L) | 11.0 (7.8-15.0) | 12.0 (9.0-17.0) | 14.0 (10.0-20.0) | 16.5 (10.8-25.0) | 18.0 (12.0-28.8) | <0.001 | 0.27* |
| γGT (U/L) | 27.0 (21.0-37.9) | 30.0 (22.0-45.0) | 34.8 (24.2-56.0) | 44.0 (28.6-82.0) | 56.8 (31.7-141) | <0.001 | 0.32* |
| Alcaline phosphatase (U/L) | 79.1 (66.6-94.3) | 84.5 (71.0-101) | 90.0 (75.8-106) | 97.9 (80.9-117) | 108 (89.9-131) | <0.001 | 0.33* |
| Lactate dehydrogenase (U/L) | 126 (111-143) | 131 (116-146) | 135 (119-153) | 136 (120-155) | 142 (125-164) | <0.001 | 0.24* |
| Bilirubine (μmol/L) | 9.0 (7.5-11.7) | 9.8 (7.9-12.0) | 10.0 (8.0-13.1) | 10.3 (8.0-14.5) | 10.3 (8.9-13.1) | <0.001 | 0.16* |

Values are expressed as median (interquartile range).
Statistical comparisons between the five sCD163 percentile categories were made using trend test.
Spearmans rho was calculated on unadjusted plasma sCD163.
*P < 0.00001.

TABLE 4
Risk of diabetes and fatty liver disease as a function of sCD163 percentile groups in the general population.

| | | Incidence rate | | | | |
|---|---|---|---|---|---|---|
| sCD163 | No. of | (95% CI) per | Age and sex | | Multifactorial | |
| percentile | participants | 10.000 person- | adjusted HR | Trend | adjusted HR | Trend |
| groups | (%)* | years | (95% CI) | P-value | (95% CI) | P-value |
| Type 2 diabetes | | | | | | |
| (N = 511) | | | | | | |
| 0-33% | 2,928 | 22 (18-27) | 1 | <0.001 | 1 | <0.001 |
| 34-66% | 2,863 | 32 (27-38) | 1.6 (1.1-2.1) | | 1.3 (0.9-1.8) | |
| 67-90% | 2,069 | 61 (52-71) | 3.4 (2.5-4.5) | | 2.3 (1.7-3.2) | |
| 91-95% | 424 | 92 (69-120) | 5.4 (3.7-8.1) | | 3.3 (2.2-4.9) | |
| 96-100% | 410 | 129 (100-164) | 7.9 (5.5-11.5) | | 5.0 (3.4-7.4) | |
| Fatty liver | | | | | | |
| disease (N = 136) | | | | | | |
| 0-33% | 2,939 | 5.9 (3.7-9.0) | 1 | <0.001 | 1 | <0.001 |
| 34-66% | 2,900 | 6.1 (3.8-9.3) | 1.4 (0.5-2.1) | | 0.9 (0.5-2.8) | |
| 67-90% | 2,103 | 14 (9.5-19) | 2.7 (1.4-5.0) | | 2.5 (1.3-4.8) | |
| 91-95% | 439 | 36 (22-56) | 8.4 (4.0-17.4) | | 7.0 (3.2-15.0) | |
| 96-100% | 418 | 97 (70-131) | 26.4 (14.3-48.5) | | 21.8 (11.4-41.8) | |

*Non-incident events were excluded, leaving 8,694 and 8,799 individuals for Cox regression analysis of type 2 diabetes and fatty liver disease, respectively. In Cox regression models age was adjusted for by incorporating age in the baseline hazard function (left truncation). In multifactorial adjusted Cox regressions, numbers vary slightly according to availability of data. Multifactorial adjustment included age (left truncation), sex, smoking, physical inactivity, body mass index, alcohol consumption, systolic blood pressure and diastolic blood pressure. HR = hazard ratio; CI = confidence interval. For the fatty liver disease group, all benign non-malignant, non-infectious liver disease diagnose codes were used (ICD8: codes 570, 5710, 5711, 5719, and 5730; ICD10: codes K70-K74, and K76K). These codes represent fatty liver disease (ref fra HJM).

### CONCLUSIONS

Elevated levels of sCD163 predict increased risk of type 2 diabetes and fatty liver disease in the general population.

Sequence listing

SEQ ID NO. 1

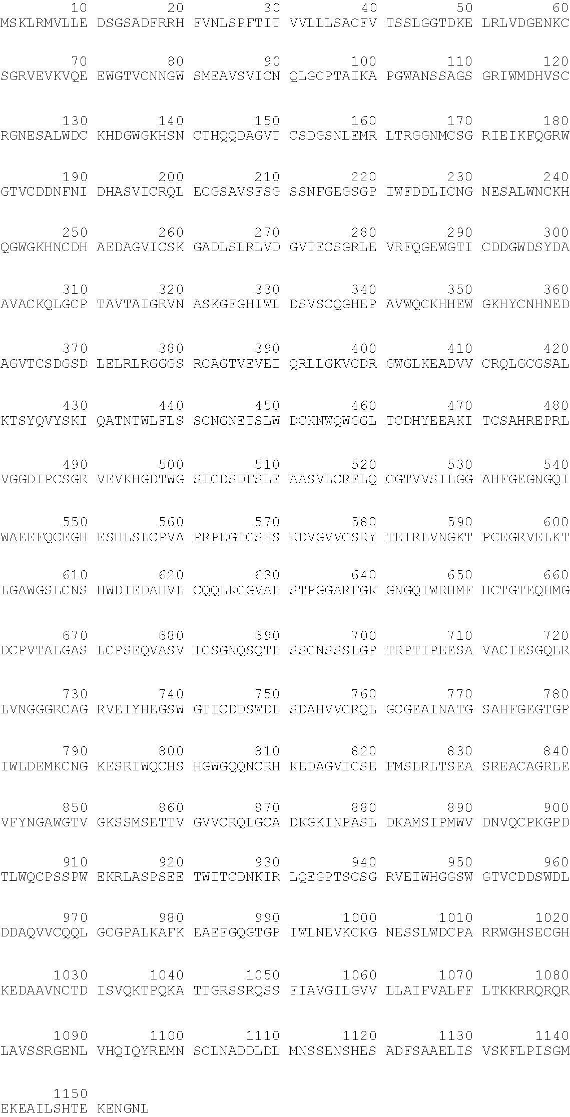

1-50. (canceled)

51. A method for assessing the likelihood of contracting a disorder, said method comprising determining the amount of soluble CD163 in a biological sample from an individual wherein a high level of soluble CD163 is indicative of an increased likelihood.


SEQUENCE LISTING (1 sequence)

SEQ ID NO: 1 - protein, 1156 aa, Homo sapiens
  SIGNAL (1)..(41)
  MISC_FEATURE (42)..(1050): Extracellular part
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAVWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL